(19) United States
(12) Patent Application Publication
(10) Pub. No.: US 2006/0287666 A1
(43) Pub. Date: Dec. 21, 2006

(54) Apparatus and methods for endoluminal advancement

(75) Inventors: Vahid Saadat, Saratoga, CA (US); Eugene Chen, Carlsbad, CA (US); Tung Thanh Le, Tustin, CA (US); Tracy D. Maahs, Rancho Santa Margarita, CA (US); Richard C. Ewers, Fullerton, CA (US); John A. Cox, Macungie, PA (US); Chris Rothe, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: USGI Medical Inc., San Clemente, CA (US)

(21) Appl. No.: 11/154,168

(22) Filed: Jun. 15, 2005

(51) Int. Cl. A61M 29/00

(52) U.S. Cl. 606/198

(57) ABSTRACT

Apparatus and methods for endoluminal advancement are described herein. A shape-lockable tissue anchoring assembly generally has an elongate body, a handle assembly, and an anchoring assembly positioned at or proximal to a distal tip of the elongate body. A distal portion of the elongate body may optionally be steerable or curvable. The anchoring assembly may include various expandable or projecting anchoring features to contact and retain tissue relative to the elongate body such that pleated tissue is temporarily immobile relative to the elongate body. This anchoring can be actuated simultaneously with or independently from shape-locking of elongate body. The anchoring assembly can be actuated simultaneously with the shape-locking of the elongate body. Alternatively, the steerable distal portion of the elongate body can be angled against the pleated tissue to retain it while the endoscope is advanced relative to the pleated tissue.

### BACKGROUND OF THE INVENTION

### Field of the Invention

The present invention relates to apparatus and methods for endoluminal advancement through a hollow body organ. More particularly, the present invention relates to apparatus and methods for achieving endoluminal access via anatomical pleating of tissue.

A physician performing a gastrointestinal examination or treatment commonly advances an endoscope through a patient's anus into the patient's colon. In order to permit full examination of the colon, the endoscope must be advanced up to the cecum. Advancement may be directed via a steerable distal end portion of the endoscope. However, at bends in the colon, e.g., at the sigmoid and especially at the two colonic flexures, advancement problems regularly occur, including a risk of injury, pain to the patient, cramp-like contractions of the colon, and even an inability to further advance the colonoscope. Much of these problems occur because the colon is comprised of soft tissue which is weakly adhered to the abdomen.

After a first deflection of the endoscope, a principal direction of force by which the endoscope is advanced no longer points towards the distal end of the endoscope, but rather points towards the readily yielding wall of the colon. Because of the severe bends typically found in the colon, as well as in the small intestines, and further because of the looping which may occur in the colon and small intestines, pleating or “accordionizing” of the tissue may be performed to facilitate advancement of the endoscope is generally along the length of the scope. This is most likely to occur if the scope is repeatedly advanced and withdrawn.

Pleating of the tissue typically enables examination of the greatest length of colon with the least amount of scope. In contrast to techniques where the scope is advanced up into the colon, accordionization typically brings the colon down over the scope. Accordionization of the tissue requires significant skill and experience on the part of the physician in order to be successful. Furthermore, many variables must be taken into account in order to properly pleat the colon, including cyclic rhythm, amount of torque, degree of tip deflection, and shaft advancement distance.

Moreover, even when the tissue has been pleated over the scope, further advancement of the scope relative to the pleated tissue may inadvertently unravel at least some of the pleating. The unraveled tissue may thereby require repeated pleating over the endoscope thereby increasing the time required for examination and also increasing the skill level necessary to complete advancement of the endoscope within the patient.

In view of the aforementioned limitations, it would be desirable to provide methods and apparatus for pleating hollow body organs, such as the colon and small intestines, that require less skill and experience on the part of the physician. It also would be desirable to provide apparatus and methods that simplify and expedite pleating of tissue.

### BRIEF SUMMARY OF THE INVENTION

An example of shapelock anchoring assembly may generally comprise an elongate body which defines at least one lumen therethrough for advancement of an endoscope or other endoscopic instruments therethrough. An anchoring assembly may be positioned at or proximal to a distal tip of the elongate body and the handle assembly may be coupled to a proximal end of the elongate body. The handle assembly may be comprised generally of a handle body and locking handle which may be configured to actuate one or more cables routed throughout the elongate body such that a plurality of nested links comprising body are compressed against one another to transition the elongate body from a flexible state to a rigid shape-locked state.

Once in its shape-locked condition, the elongate body maintains any configuration in a rigid manner. Release of the locking handle relative to handle body releases the elongate body to transition back into a flexible body to conform into another configuration. An endoscope or any number of endoscopic instruments may be advanced into and through an entry lumen and elongate body to effect treatment. Further details and examples of shape-locking elongate bodies are disclosed in U.S. patent application Ser. No. 10/281,462 filed Oct. 25, 2002 (U.S. Pat. Pub. 2003/0233066 A1), which is incorporated herein by reference in its entirety.

In addition to the shape-locking features of the elongate body, the anchoring assembly may comprise, in one variation, an expandable or projected anchoring feature which may be utilized to contact and anchor or retain tissue relative to the elongate body such that the anchored tissue is temporarily immobile relative to the elongate body. In one variation, the anchoring assembly comprises an anchoring member attached at its proximal end to the elongate body via a proximal band. The distal portion of a mesh member may be attached to a distal band which may be movable relative to the elongate body. The mesh member may be reconfigurable from a low-profile configuration for advancement within the patient body to a radially expanded anchoring configuration. The mesh member may be expanded in this variation by urging the distal band relatively towards the proximal band to compress the mesh member therebetween and to thereby radially expand the member. In alternative variations, the proximal band may be advanced distally towards a static distal band or both bands may be translated towards one another simultaneously to effect an anchoring configuration.

The anchoring assembly may likewise be actuatable such that actuation of the mechanism for the shape-locking feature of the elongate body simultaneously actuates expansion of, in this variation, the mesh member. Alternatively, the anchoring assembly may be actuated via a separate mechanism such that shape-lock actuation of the elongate body and anchor actuation remain independent from one another.

One variation for simultaneous shape-lock and anchor actuation may comprise at least one or more actuation wires attached to the distal band at attachment points and routed beneath or through a liner within respective lumens along elongate body and attached at corresponding attachment points located on a shape-locking actuation shaft extending from the handle. At least one actuation wire is used and several wires uniformly positioned about a circumference of distal band may also be utilized. Upon depressing the locking handle to shape-lock elongate body, the attachment points may be pulled proximally towards the handle body such that actuation wires likewise pull the distal band proximally relative to the proximal band, which is statically attached to the elongate body. The approximation of the proximal and distal bands towards one another compresses the anchoring member such that the mesh member is forced to radially expand and come into contact with the tissue to be temporarily anchored. Upon releasing the locking handle, the actuation wires may slacken to allow for the mesh member to reconfigure itself back into its original low-profile shape.

Additional methods and mechanisms which may be utilized in combination with a shape-lockable elongate body may be seen in further detail in U.S. patent application Ser. No. 10/746,286 filed Dec. 23, 2003 (U.S. Pat. Pub. 2004/0186349 A1), which is incorporated herein by reference in its entirety. Any of the tissue anchoring variations may be actuated via a mechanism common to the shape-locking feature and anchoring feature such that a single operation may actuate both shape-locking and tissue anchoring features simultaneously. Alternatively, the anchoring feature may also be actuated independently of the shape-locking feature, if so desired by the user.

Additional tissue anchoring mechanisms and methods may include one or more expandable balloons disposed near or at the distal end of the elongate body and expandable into a variety of configurations, expandable balloon members having a covering or coating disposed over the outer surface of the balloon, as well as one or more expandable balloon members which are configured to expand into an eccentric shape relative to a longitudinal axis of the elongate. Other tissue anchoring variations may include pivoting members which lie against the elongate body in a low-profile configuration and rotates from a proximal-to-distal or distal-to-proximal pivoting manner like a clamshell into its anchoring configuration, malecot-type anchoring assemblies, distensible sheaths or membranes supported via one or several radially extending support members, compliant sleeves or portions having a plurality of projections which may extend from a low-profile shape to an extended configuration, as well as portions of the elongate body having a compressible element, such as a compressible spring elements, having an expandable covering or coating, e.g., mesh covering.

In an example of use for pleating and/or anchoring tissue, one method may include advancing a conventional endoscope into a colon of a patient body along with an elongate body of the shape-lockable device in its flexible state and having an expandable anchor disposed thereon. Once the shape-lockable elongate body has been desirably advanced over the endoscope body, the steerable distal section of the endoscope may be steered into a hooked configuration to engage the colon tissue. The hooked and engaged tissue may be actively pulled proximal relative to the elongate body such that a portion of the colon is shortened by the engaged tissue being drawn and pleated over the elongate body. Once the endoscope is to be further advanced through the colon, the anchor member on the elongate body may be actuated to expand and thereby capture the pleated tissue along elongate body, prior to advancement of the endoscope or shortly after advancement of the endoscope relative to the elongate. The elongate body may also be optionally actuated to shape-lock its configuration simultaneously with anchor expansion or sequential to anchor expansion.

With the pleated tissue captured and inhibited from unraveling by the anchoring member, and with the elongate body optionally shape-locked (if so desired), the steerable distal portion of the endoscope may be straightened from its hooked configuration and the endoscope body may be advanced further into the colon relative to the captured pleated tissue. Upon further advancing the endoscope, the steerable distal portion may again be formed into a hooked configuration for hooking and pulling the tissue engaged thereby. Again, tissue engaged by the steerable distal portion of the endoscope may be drawn proximally towards the elongate body and the anchoring member, which may be reconfigured into its low-profile to allow for distal advancement of elongate body. If the elongate body were optionally shape-locked, it may be transitioned into its flexible configuration as well.

Once the anchoring member has been released from tissue engagement, further pleating of colon tissue around the elongate body may be accomplished. With the elongate body advanced distally further into the colon and colon tissue further pleated about the elongate body, the anchoring member may be actuated again to capture the additional pleated tissue and prevent its unraveling as the endoscope steerable distal portion and endoscope body are again advanced distally further through the colon. This process may be repeated as many times as desired or necessary to shorten the colon as well as to remove any tortuous looping of portions of the colon and to facilitate advancement of an endoscope or endoscopic instruments therethrough.

In another method for endoscope advancement and tissue pleating, a shape-lockable elongate body may additionally comprise a shape-lockable distal steerable portion. In use, a steerable endoscope and shape-lockable elongate body may be advanced into a patient's colon, as described above. The steerable distal portion of the endoscope may be steered into a hooking configuration to engage a portion of the colon which may then be pulled proximally to pleat the tissue about the elongate body in a hook-and-pull maneuver. To prevent the pleated tissue from unraveling, the steerable portion of the elongate body may be urged into an angled, arcuate, or curved configuration such that steerable portion is curved relative to a longitudinal axis of a proximal portion of the elongate body. The steerable portion may be simply angled or curved such that the steerable portion rests against a portion of the colon along a tissue contact region. Thus, the tissue contact region between the curved distal portion and the colon may be sufficient to anchor the pleated tissue and prevent unraveling as the steerable distal portion of the endoscope is straightened and endoscope body is further advanced into the colon.

### BRIEF DESCRIPTION OF THE DRAWINGS

FIGS. 1A and 1B illustrate an elongate shape-locking device transitioning from a flexible state to a rigid state, respectively, with a simultaneously actuatable tissue anchoring or adhering mechanism.

FIGS. 1C and 1D illustrate an end view of the elongate device from FIGS. 1A and 1B, respectively, having the actuatable tissue anchoring or adhering mechanism in a low profile and extended configuration.

FIGS. 2A and 2B show one example of actuation of the tissue anchoring or adhering mechanism upon actuation of the shape-locking feature of the elongate device.

FIGS. 3A to 3C show another variation for an anchoring or adhering mechanism in low-profile and expanded side views and an expanded end view, respectively.

FIGS. 4A to 4C show yet another variation for an anchoring or adhering mechanism in low-profile and expanded side views and an expanded end view, respectively.

FIGS. 5A to 5C show yet another variation for an anchoring or adhering mechanism in low-profile and expanded side views and an expanded end view, respectively.

FIGS. 6A to 6C show yet another variation for an anchoring or adhering mechanism in low-profile and expanded side views and an expanded end view, respectively.

FIGS. 7A to 7C show yet another variation for an anchoring or adhering mechanism in low-profile and expanded side views and an expanded end view, respectively.

FIGS. 8A to 8C show yet another variation having an expandable balloon with a covering or coating, such as mesh, disposed thereupon.

FIGS. 9A to 9C show yet another variation having one or more expandable members, such as balloons, which may be off-set relative to a longitudinal axis of the elongate body.

FIGS. 10A to 10C show yet another variation in which an expandable member may be configured into a helical configuration.

FIGS. 11A to 11C show yet another variation where a plurality of projections may be configured to expand or extend at an angle to allow for uni-directional travel of the elongate body through tissue.

FIGS. 12A to 12C show yet another variation utilizing a compressible member, such as a spring, to expand a mesh anchor.

FIGS. 13A to 13D illustrates another variation for facilitating endoscope advancement utilizing a shape-locking system in which one or both shape-locking devices may utilize a tissue anchoring or adhering mechanism.

FIGS. 14A to 14H illustrates a method for advancing an endoscope through a colon of a patient in combination with a shape-lockable device configured to temporarily retain pleated portions of the tissue to facilitate endoscope advancement.

FIGS. 15A to 15F illustrates another method for advancing an endoscope through a colon utilizing a shape-lockable device configured to angle a distal portion thereof against the tissue to retain pleated portions while advancing an endoscope distally.

### DETAILED DESCRIPTION OF THE INVENTION

Generally in use, an endoscope may be advanced into a patient's body lumen, such as the lower gastro-intestinal tract via the anus or the upper gastro-intestinal tract via the patient's mouth. However, the tissue of the colon and small intestines are typically unsupported and advancement through these body lumens is difficult. Furthermore, looping of the tissue and unraveling of pleated tissue relative to the endoscope makes endoscopic advancement particularly difficult. Accordingly, secured or temporarily anchored accordionization or pleating of the tissue around the endoscope may facilitate advancement of the endoscope and examination of the tissue.

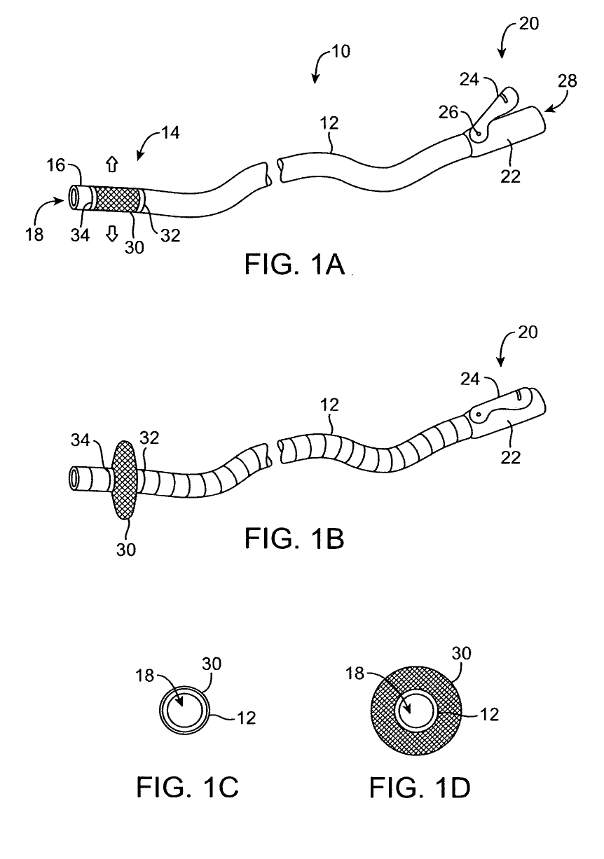

An example of a device is shown in shapelock anchoring assembly 10 in FIGS. 1A and 1B. Shapelock anchoring assembly 10 may generally comprise an elongate body 12 which defines at least one lumen 18 therethrough for advancement of an endoscope or other endoscopic instruments therethrough. Anchoring assembly 14, described further below, may be positioned at or proximal to distal tip 16 of elongate body 12 and handle assembly 20 may be coupled to a proximal end of elongate body 12.

Handle assembly 20 may be comprised generally of handle body 22 and locking handle 24 which may be configured to actuate one or more cables routed throughout elongate body 12 such that a plurality of nested links comprising body 12 are compressed against one another to transition elongate body 12 from a flexible state, shown in FIG. 1A, to a rigid shape-locked state, shown in FIG. 1B. Once in its shape-locked condition, elongate body 12 maintains any configuration in a rigid manner. Release of locking handle 24 relative to handle body 22 releases elongate body 12 to transition back into a flexible body to conform into another configuration.

Locking handle 24 may be rotatably coupled to handle body 22 via pivot 26 such that rotation of locking handle 24 in the direction shown in FIG. 1A against handle body 22 may actuate the shape-locking feature of elongate body 12. However, any number of actuation mechanisms as generally known may also be utilized. Handle body 22 may also define in its proximal end an entry lumen 28 which extends through handle assembly 20 and elongate body 12. As mentioned above, an endoscope or any number of endoscopic instruments may be advanced into and through entry lumen 28 and elongate body 12 to effect treatment through assembly 10. Further details and examples of shape-locking elongate bodies are disclosed in U.S. patent application Ser. No. 10/281,462 filed Oct. 25, 2002 (U.S. Pat. Pub. 2003/0233066 A1), which is incorporated herein by reference in its entirety.

In addition to the shape-locking features of elongate body 12, anchoring assembly 14 may comprise, in one variation, an expandable or projected anchoring feature which may be utilized to contact and anchor or retain tissue relative to elongate body 12 such that the anchored tissue is temporarily immobile relative to elongate body 12. In the variation shown in FIGS. 1A and 1B, anchoring assembly 14 comprises an anchoring member, shown here as reconfigurable mesh member 30, attached at its proximal end to elongate body 12 via proximal band 32. The distal portion of mesh member 30 may be attached to a distal band 34 which may be movable relative to elongate body 12.

Mesh member 30 may be reconfigurable from a low-profile configuration, shown in FIG. 1A, for advancement within the patient body to a radially expanded anchoring configuration, shown in FIG. 1B. Mesh member 30 may be expanded in this variation by urging distal band 34 relatively towards proximal band 32 to compress mesh member 30 therebetween and to thereby radially expand member 30. In alternative variations, proximal band 32 may be advanced distally towards a static distal band 34 or both bands 32, 34 may be translated towards one another simultaneously to effect an anchoring configuration. FIGS. 1C and 1D show end views of elongate body 12 with mesh member 30 in its low-profile configuration and its expanded configuration, respectively.

Anchoring assembly 14 may likewise be actuatable via handle assembly 20 such that actuation of the mechanism for the shape-locking feature of elongate body 12 simultaneously actuates expansion of, in this variation, mesh member 30. Alternatively, anchoring assembly 14 may be actuated via a separate mechanism, e.g., control wheel or slider mechanism, such that shape-lock actuation of elongate body 12 and anchor actuation remain independent from one another.

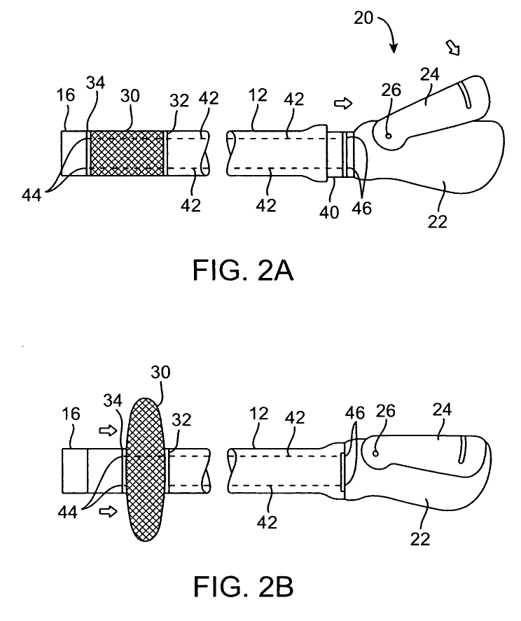

One variation for simultaneous shape-lock and anchor actuation is shown in the side views of FIGS. 2A and 2B. In this variation, at least one or more actuation wires 42 may be attached to distal band 34 at attachment points 44 and routed beneath or through a liner within respective lumens along elongate body 12 and attached at corresponding attachment points 46 located on a shape-locking actuation shaft 40 extending from handle 22. At least one actuation wire 42 is used and several wires uniformly positioned about a circumference of distal band 34 may also be utilized. Upon depressing locking handle 24 to shape-lock elongate body 12, attachment points 46 may be pulled proximally towards handle body 22 such that actuation wires 42 likewise pull distal band 34 proximally relative to proximal band 32, which is statically attached to elongate body 12 and as shown in FIG. 2B. The approximation of proximal and distal bands 32, 34, respectively, towards one another compresses the anchoring member, in this variation mesh member 30, such that mesh member 30 is forced to radially expand and come into contact with the tissue to be temporarily anchored. Upon releasing locking handle 24, the actuation wires 42 may slacken to allow for mesh member 30 to reconfigure itself back into its original low-profile shape.

Anchoring assembly 14 may be comprised of a number of different anchoring configurations and mechanisms. Mesh member 30 is shown as an example of one type of tissue anchoring variation. Additional methods and mechanisms which may be utilized in combination with a shape-lockable elongate body 12 may be seen in further detail in U.S. patent application Ser. No. 10/746,286 filed Dec. 23, 2003 (U.S. Pat. Pub. 2004/0186349 A1), which is incorporated herein by reference in its entirety.

Any of the tissue anchoring variations described herein as well as in U.S. patent application Ser. No. 10/746,286 may be actuated via a mechanism common to the shape-locking feature and anchoring feature such that a single operation, such as depressing locking handle 24, may actuate both shape-locking and tissue anchoring features simultaneously. Alternatively, the anchoring feature may also be actuated independently of the shape-locking feature, if so desired by the user.

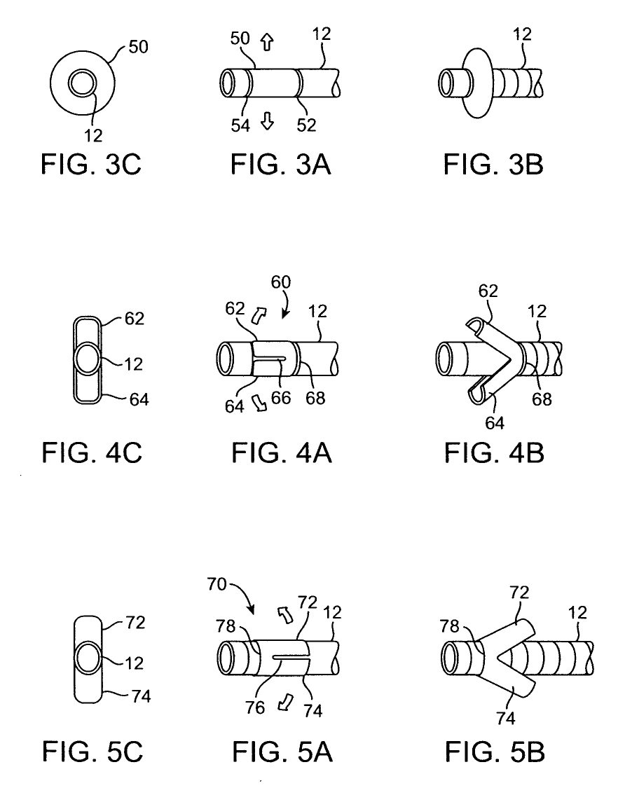

Turning to FIGS. 3A to 3C, additional tissue anchoring mechanisms and methods are shown. As aforementioned, this and other anchoring variations described herein may be commonly actuated upon shape-locking the elongate body 12 or actuated independently from shape-locking. As shown in the figures, elongate body 12 may comprise an expandable balloon 50 disposed near or at the distal end of elongate body 12, as shown in the side view of FIG. 3A. Balloon 50 may be attached to elongate body 12 via proximal and distal attachment regions 52, 54, respectively, and fluidly coupled via an inflation lumen routed through elongate body 12. When inflated, as shown in the respective side and end views of FIGS. 3B and 3C, balloon 50 may expand radially to contact the tissue to be temporarily anchored. Balloon 50 may be made from a variety of biocompatible materials as conventionally known.

In yet another variation, elongate body 12 may utilize a pivoting anchoring assembly 60 generally comprising a first and a second pivoting member 62, 64, respectively, which lie against elongate body in a low-profile configuration, as shown in FIG. 4A, and rotates from a proximal-to-distal pivoting manner like a clamshell into its anchoring configuration, as shown in respective side and end views in FIGS. 4B and 4C. Pivoting members 62, 64 may define a slot 66 separating the two in the low-profile configuration and they may be attached to elongate body via proximal attachment portion 68. Moreover, pivoting members 62, 64 may be biased via attachment portion 68 to lie in its low-profile shape against elongate body 12 until urged open via pull wires attached to respective members 62, 64. Pivoting members 62, 64 may be formed into a variety of shapes provided that the outer edges contacting the tissue are atraumatic. Moreover, members 62, 64 may be formed from various materials which are generally compliant and flexible, such as low durometer polymeric materials or flexible superelastic alloys. Furthermore, although two pivoting members 62, 64 are shown in this variation, any number of pivoting members uniformly or asymmetrically positioned circumferentially about elongate body 12 as practicable may be utilized.

The variation of pivoting anchor assembly 70 shown in the side and end views of FIGS. 5A to 5C are similar to the variation shown in FIGS. 4A to 4C; however, the pivoting members 72, 74, which are separated by slot or boundary 76, are attached at its distal end via attachment portion 78 such that pivoting members 72, 74 expand from its low-profile configuration to its anchoring configuration by rotating from a distal-to-proximal manner, as shown in FIGS. 5A and 5B.

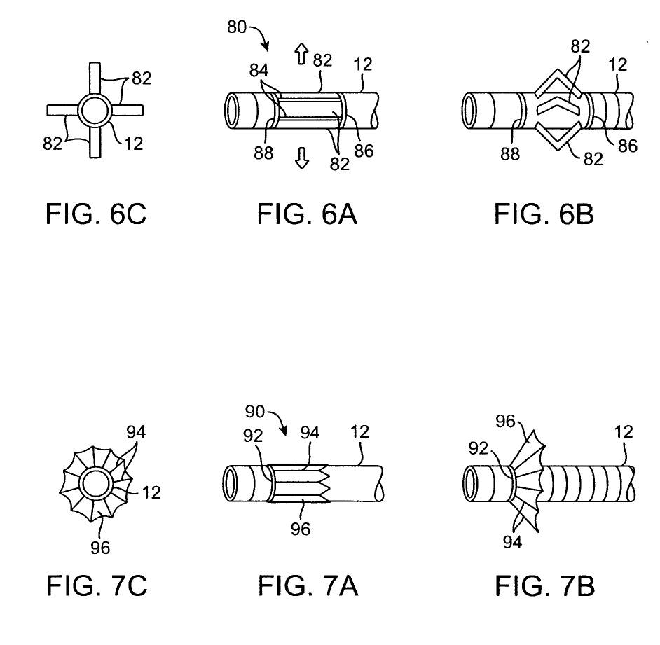

In another variation, malecot anchoring assembly 80 shown in FIGS. 6A to 6C may comprise generally one or several pivoting arm members 82 which reconfigure from a low-profile shape to a pivoted malecot assembly when actuated. pivoting arm members 82 may be connected at their proximal and distal ends at proximal and distal attachment bands 86, 88, respectively. Moreover, each of the arms 82 may be separated from one another via slots or boundaries 84. In operation, actuation wires may tension to pull distal attachment band 88 proximally with respect to proximal attachment band 86 until arms 82 are forced to pivot into its anchoring configuration, shown in FIG. 6B and the end view of FIG. 6C. Each of the arms 82 may be preformed to pivot into various extended configurations and they may also be biased to collapse into a low-profile. Accordingly, arms 82 may be formed from a variety of materials as described above.

In yet another variation, expandable anchor assembly 90 may generally comprise a distensible sheath or membrane 96 which may be supported via one or several radially extending support members 94. Each of the support members 94, as well as the sheath or membrane 96, may be secured at their distal ends to elongate body 12 along a distal anchoring portion 92. In use, support members 94 may be extended radially, as shown in the respective side and end views of FIGS. 7B and 7C, such that sheath or membrane 96 is expanded or extended for contact against tissue.

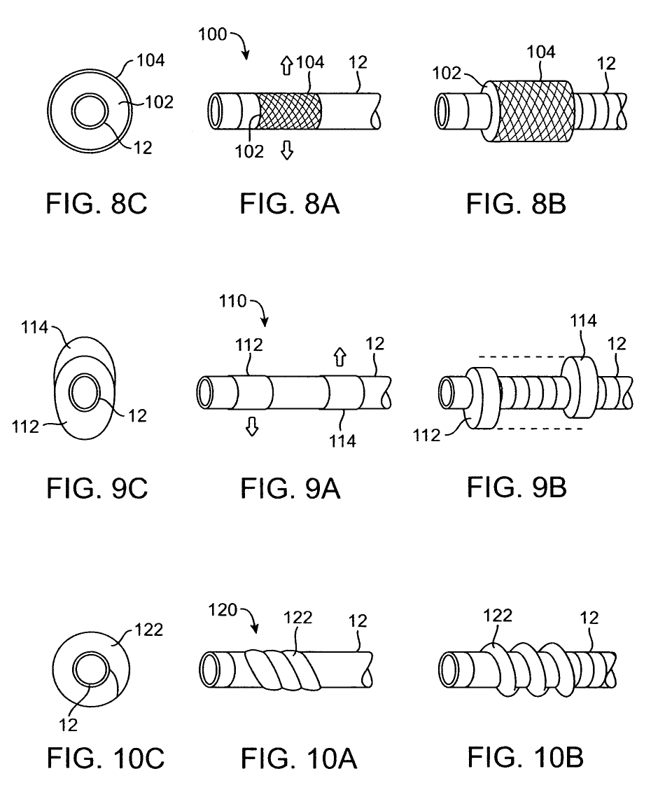

In another variation shown in FIGS. 8A to 8C, alternative anchoring assembly 100 may generally comprise an expandable balloon member 102 disposed near or at the distal end of elongate body 12 with a covering or coating disposed over the outer surface of balloon 102. In this example, the covering or coating may comprise a mesh covering 104 attached or adhered to the balloon outer surface to provide increased frictional resistance for enhanced anchoring against tissue, as shown in FIGS. 8B and 8C. Balloon 102 may be fabricated from any number of distensible or expandable materials as generally known and it may optionally be configured to expand to a pre-determined radius. Mesh covering 104 may comprise any number of expandable or woven materials which are configured to have a greater frictional resistance to slippage with respect to tissue. Upon expansion of balloon 102, mesh covering 104 may likewise be expanded by balloon 102 for contact against tissue and then upon balloon 102 retracting back into its low-profile configuration, mesh covering 104 may likewise retract with balloon 102.

In yet another variation, elongate body 12 may comprise one or more expandable balloon members, as shown in FIG. 9A, which are configured to expand into an eccentric shape relative to a longitudinal axis of elongate body 12. As shown in the respective side and end views of FIGS. 9B and 9C, off-set balloon assembly 110 may comprise a first off-set balloon 112 and second off-set balloon 114 which are positioned upon elongate body 12 and placed at a distance from one another. When expanded, off-set balloons 112, 114 may reconfigure into expanded configurations which are off-set relative to one another, as well as relative to elongate body 12. This expanded off-set configuration may conform a portion of elongate body 12 into a curved or bent section with respect to the tissue to facilitate maintenance of tissue anchoring with respect to elongate body 12. Although two off-set balloons are shown in the figures, this is merely exemplary of the off-set balloon configuration and a single off-set balloon may be utilized or more than two off-set balloons set in various configurations may also be utilized.

In another variation, a helical balloon assembly 120 may generally comprise an expandable balloon configured to maintain a low-profile shape, as in FIG. 10A, and expand to form a helical balloon 122 over elongate body 12, as in FIGS. 10B and 10C. Any number of turns and various pitches of helical balloon 122 may be incorporated into its configuration, as desired.

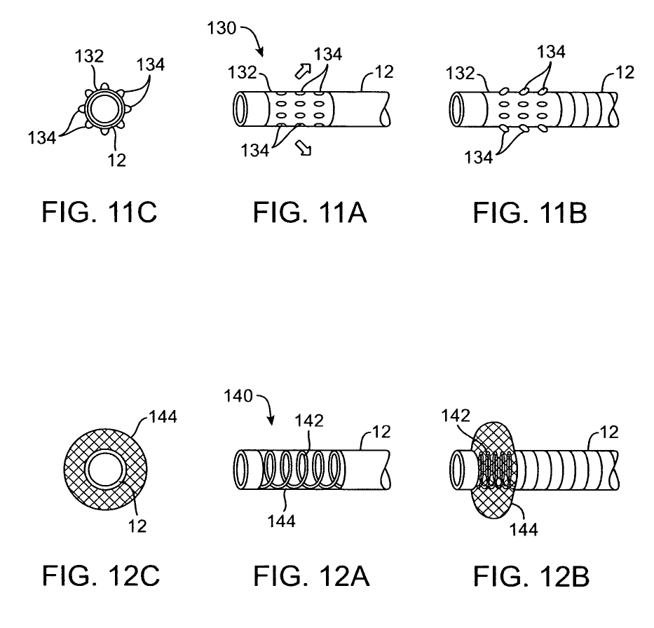

FIGS. 11A to 11C show respective side and end views of a configuration having an expandable projection assembly 130. In this variation, sleeve 132 may generally comprise a compliant sleeve or portion having a plurality of projections which may extend from a low-profile shape, as in FIG. 11A, to an extended configuration, as in FIGS. 11B and 11C. The projections 134 may be configured to extend at a perpendicular angle relative to a longitudinal axis of elongate body 12 for tissue anchoring; alternatively, projections 134 may be configured to extend at an angle, as shown in the figures, which project proximally to provide for enhanced tissue anchoring. Angled projections 134 may be left in its expanded configuration during advancement of elongate body 12 in the patient to provide for uni-directional advancement of elongate body 12 such that distal advanced may be accomplished, as usual, but proximal withdrawal of elongate body 12 or tissue pleated over angled projections 134 within the hollow body organ is inhibited from unraveling.

Angled projections 134, as well as sleeve 132, may be fabricated from a distensible material such that projections 134 lie flat relative to elongate body 12 but distend when inflated or expanded, e.g., via saline, water, nitrogen, carbon dioxide, etc. Alternatively, projections 134 may be configured to bias to form an inverted configuration within sleeve 132 to provide for a low-profile during advancement of elongate body 12. When inflated or expanded, the inverted angled projections 134 may evert to extend into its expanded, angled configuration. Moreover, any number of projections 134 may be utilized in a number of configurations. They may be uniformly positioned about sleeve 132, as shown in the figures, or they may alternatively be positioned in single or multiple circumferential patterns; alternatively, projections 134 may be positioned in one or more linear or non-linear pattern along the length of sleeve 132.

In yet another variation, a spring body assembly 140 may be utilized to expanded or project an anchoring mechanism. As shown in FIG. 12A, elongate body 12 may comprise at least one portion of the body having a compressible element, such as a compressible spring element 142. The portion of elongate body 12 containing spring element 142 may have the spring replace a portion of the body such that spring element 142 has a diameter which approximates the outer surface of elongate body 12. The spring element 142 may have a covering or coating, e.g., mesh covering 144, which is attached at its proximal end to elongate body 12 and its distal end attached to the portion distal of spring element 142.

When in its non-compressed state, spring element 142 may stretch the mesh covering 144 into a low-profile configuration. However, when a distal portion of spring element 142 is drawn proximally towards elongate body 12, e.g., by actuation wires attached to a distal portion of spring 142 or to a distal tip of elongate body 12 being tensioned from their proximal ends outside the patient, spring element 142 may become compressed and expand the mesh covering 144 into its expanded anchoring profile, as shown in the respective side and end views of FIGS. 12B and 12C. When the tensioning wires are released or relaxed, spring element 142 may expand into its relaxed profile to reconfigure mesh covering 144 back into its low-profile configuration.

As mentioned above, any of the tissue anchoring variations described herein may be actuated via a mechanism common to the shape-locking feature and anchoring feature such that a single operation actuates both shape-locking and tissue anchoring features simultaneously. Alternatively, the anchoring feature may also be actuated independently of the shape-locking feature.

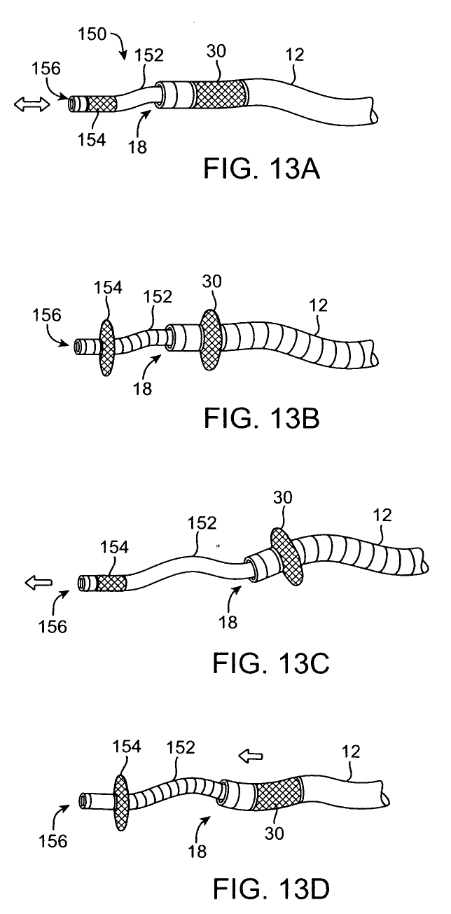

Although the tissue anchoring system disclosed may be used as a single shape-lockable elongate body 12 with an endoscope or other endoscopic instruments, multiple elongate devices which are also optionally shape-lockable may be used in combination with one another. One example is shown in the assembly of FIGS. 13A to 13D. A first shape-lockable elongate body 12 having, in this example mesh element 30 although other anchoring variations may be utilized, may be used in combination with a second shape-lockable elongate assembly 150 similarly having a shape-lockable elongate body 152 with an anchoring mechanism, e.g., anchor mesh element 154 although any of the other variations may be used herewith. The second shape-lockable assembly 150 may be passed through lumen 18 of the first elongate body 12 and second elongate body 152 may also define a working lumen 156 therethrough for the passage of endoscopes or endoscopic instruments therethrough.

In use, second elongate body 152 may be advanced relative to first elongate body 12 in an alternating manner while both bodies 12, 152 are in a relaxed, non-rigid state, as shown in FIG. 13A. Upon reaching a first location within a hollow body organ with second elongate body 152 extending at least partially distal relative to first elongate body 12, both anchoring members 30, 154 may be expanded to temporarily engage the tissue surfaces. Both elongate bodies 12, 152 may be optionally shape-locked into a rigid condition, as seen in FIG. 13B, while anchoring members 30, 154 are expanded and the engaged tissue may be pulled proximally with both anchoring members 30, 154 to pleat the engaged tissue.

After pleating the tissue, anchoring member 30 may be maintained in its expanded configuration and optionally first elongate body 12 may also be maintained in a shape-locked configuration while anchoring member 154 may be reconfigured into a low-profile and second elongate body 152 may be advanced distally relative to any pleated tissue and relative to first elongate body 12, as shown in FIG. 13C. If second elongate body 152 were optionally shape-locked, it may be reconfigured from its rigid state back into its flexible state prior to advancing second elongate body 152 relative to first elongate body 12.

Once second elongate body 152 has been desirably advanced within the patient, anchoring member 154 may be expanded to engage the distally located tissue and second elongate body 152 may optionally be shape-locked, if so desired. Anchoring member 30 may be reconfigured into its low-profile shape and then advanced over or along second elongate body 152, which may or may not be shape-locked. If first elongate body 12 were shape-locked, it is preferably transitioned into a its flexible state prior to advancement over second elongate body 152. Once first elongate body 12 has been desirably advanced, anchoring member 30 may then be expanded and elongate body 12 may be optionally shape-locked and the process repeated for further advancement within the patient. The process may be reversed for withdrawal from the patient; alternatively, both anchoring members 30, 154 may be configured into their low-profiles and both elongate bodies 12, 152 may be transitioned into their flexible shapes for withdrawal from the patient.

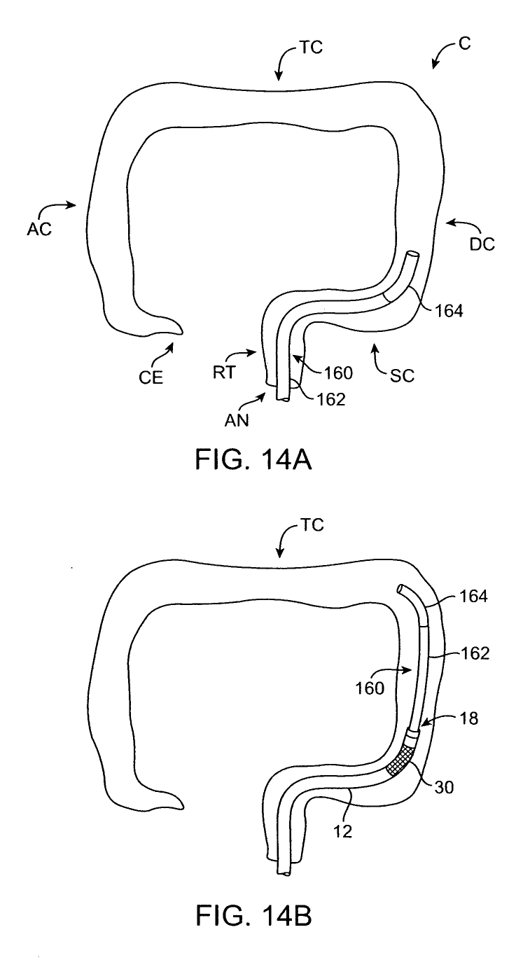

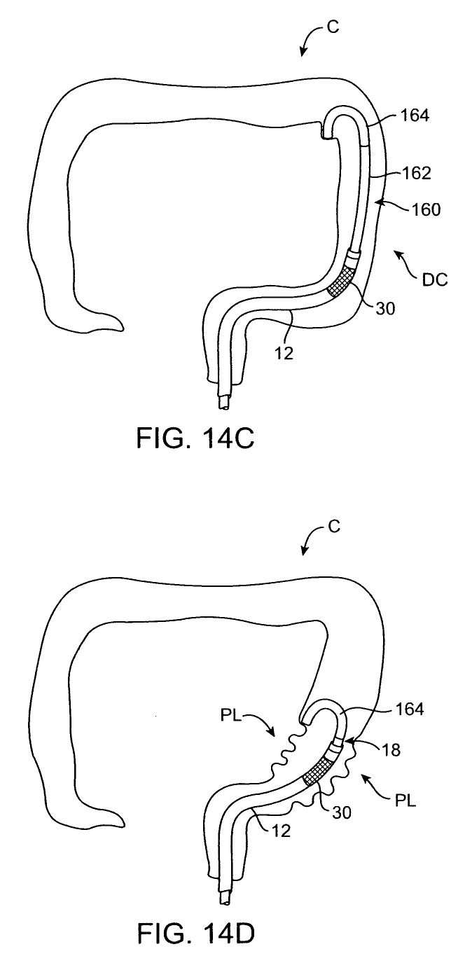

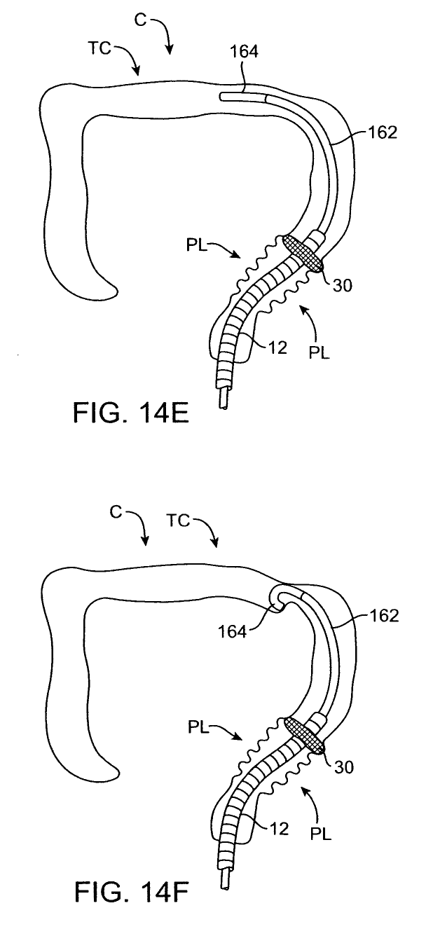

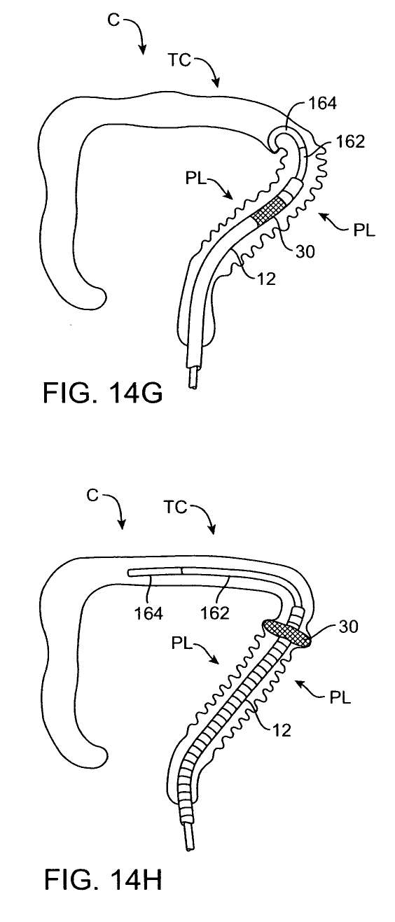

In an example of use for pleating and/or anchoring tissue, FIGS. 14A to 14H illustrate one method for device advancement and tissue pleating. As shown in FIG. 14A, a conventional endoscope 160 may be advanced into a colon C of a patient body through the anus AN and rectum RT. The descending colon DC, transverse colon TC, ascending colon AC, and cecum CE may be seen for reference. The endoscope 160 may comprise a flexible endoscope body 162 and a steerable distal section 164, as generally known in the art. Endoscope 160 may be steered through the sigmoid colon SC and partially into the descending colon DC.

Once endoscope 160 has been desirably positioned, elongate body 12 in its flexible state and having expandable anchor 30 disposed thereon, may be advanced into the colon C over or along endoscope body 162. Once shape-lockable elongate body 12 has been desirably advanced over endoscope body 162, endoscope 160 may be advanced further into colon C relative to elongate body 12, as shown in FIG. 14B. Alternatively, rather than using a conventional endoscope 160, a second shape-lockable elongate body, as described above, may be advanced relative to elongate body 12.

Once endoscope 160 has been advanced to a desirable position, the steerable distal section 164 may be steered into a hooked configuration to engage the tissue of colon C to endoscope 160, as shown in FIG. 14C. The hooked and engaged tissue may be actively pulled proximal relative to elongate body 12 such that a portion of colon C is shortened by the engaged tissue being drawn and pleated over elongate body 12, as seen in FIG. 14D. Once the endoscope 160 is to be further advanced through colon C, distally advancing the endoscope 160 may unravel at least some of the pleated tissue PL by dragging the tissue distally along with endoscope 160 and/or some of the pleated tissue PL may spontaneously unravel on its own. Accordingly, prior to advancement of endoscope 160 or shortly after advancement of endoscope 160 relative to elongate body 12, anchor member 30 on elongate body 12 may be actuated to expand and thereby capture the pleated tissue PL along elongate body 12, as shown in FIG. 14E. Elongate body 12 may also be optionally actuated to shape-lock its configuration simultaneously with anchor expansion or sequential to anchor expansion.

As seen in FIG. 14E, with pleated tissue PL captured and inhibited from unraveling by anchoring member 30, and with elongate body 12 optionally shape-locked (if so desired), steerable distal portion 164 may be straightened from its hooked configuration and endoscope body 162 may be advanced further into colon C relative to the captured pleated tissue PL. Upon further advancing endoscope 160 further to a desirable location, steerable distal portion 164 may again be formed into a hooked configuration for hooking and pulling the tissue engaged thereby, as shown in FIG. 14F.

Again, tissue engaged by steerable distal portion 164 may be drawn proximally towards elongate body 12 and anchoring member 30, which may be reconfigured into its low-profile to allow for distal advancement of elongate body 12. If elongate body 12 were optionally shape-locked, it may be transitioned into its flexible configuration as well. Once anchoring member 30 has been released from tissue engagement, further pleating of colon tissue around elongate body 12 may be accomplished, as shown in FIG. 14G. With elongate body 12 advanced distally further into colon C and colon tissue further pleated about elongate body 12, anchoring member 30 may be actuated again to capture the additional pleated tissue PL and prevent its unraveling as steerable distal portion 164 and endoscope body 162 are again advanced distally further through colon C, as shown in FIG. 14H. Elongate body 12 may also again be optionally shape-locked to facilitate advancement of endoscope body 162 through colon C. This process may be repeated as many times as desired or necessary to shorten the colon C as well as to remove any tortuous looping of portions of the colon C and to facilitate advancement of an endoscope or endoscopic instruments therethrough.

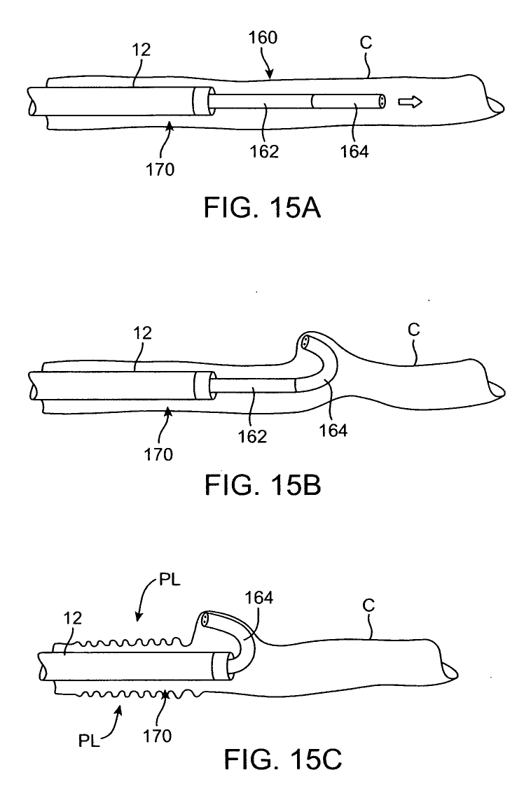

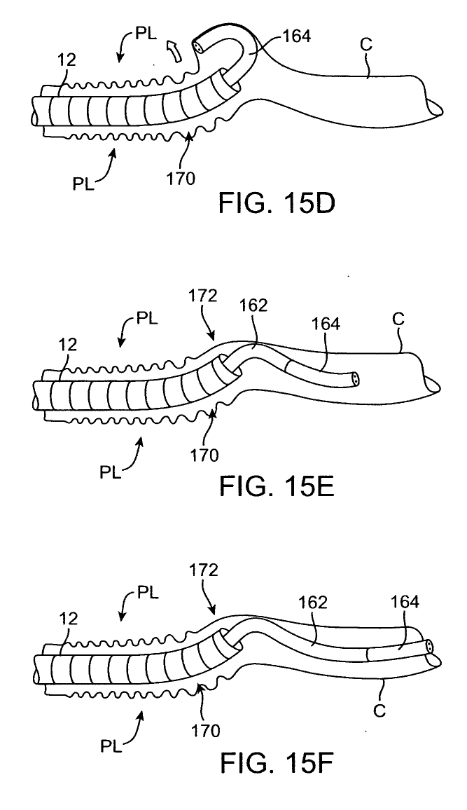

In another method for endoscope advancement and tissue pleating, a shape-lockable elongate body 12 may additionally comprise a shape-lockable distal steerable portion 170. In use, a steerable endoscope 160 and shape-lockable elongate body 12 may be advanced into a patient's colon C, as shown in FIG. 15A and as described above. Also as above, the steerable distal portion 164 of endoscope 160 may be steered into a hooking configuration to engage a portion of colon C, as shown in FIG. 15B. The engaged colon C may then be pulled proximally to pleat the tissue about elongate body 12 in a hook-and-pull maneuver, as seen in FIG. 15C.

To prevent the pleated tissue PL from unraveling, the steerable portion 170 of elongate body 12 may be urged into an angled, arcuate, or curved configuration, as shown in FIG. 15D, such that steerable portion 170 is curved relative to a longitudinal axis of a proximal portion of elongate body 12. The degree to which steerable portion 170 is curved need not be large, e.g., greater than 45°, relative to the rest of elongate body 12, although steerable portion 170 may be curved anywhere from 0° to 90° relative to the rest of elongate body 12. Steerable portion 170 may be simply angled or curved such that the steerable portion 170 rests against a portion of colon C along a tissue contact region 172, as shown in FIG. 15E. Thus, the tissue contact region 172 between curved distal portion 170 and colon C may be sufficient to anchor the pleated tissue PL and prevent unraveling as the steerable distal portion 164 of the endoscope 160 is straightened and endoscope body 162 is further advanced into colon C, as shown in FIG. 15F.

Optionally, during this hook-and-pull procedure, when steerable portion 170 is curved into its anchoring configuration, elongate body 12 may be configured to shape-lock into its rigid state simultaneously with the actuation of steerable portion 170 into its curved configuration, as seen in FIG. 15D. This may be accomplished by linking the steering and shape-locking into a common mechanism actuatable from outside the patient body. Alternatively, elongate body 12 may be shape-locked independently from actuation of steerable portion 170 and elongate body 12 may be shape-locked either before, during, or after actuation of steerable portion 170 into its curved configuration. In yet another alternative, the shape-locking of elongate body 12 may be omitted entirely and steerable portion 170 may be curved into its anchoring configuration without engaging the shape-locking feature of elongate body 12. Additionally, any of the expandable tissue anchoring mechanisms as described above may be utilized in combination with a steerable portion 170 to further enhance anchoring of the pleated tissue PL.

Although various illustrative embodiments are described above, it will be evident to one skilled in the art that a variety of combinations of aspects of different variations, changes, and modifications are within the scope of the invention. It is intended in the appended claims to cover all such changes and modifications that fall within the true spirit and scope of the invention.

What is claimed is:

1. A system for advancing through a hollow body organ, comprising:
an elongate body adapted to transition between a flexible state and a rigidized state, wherein the elongate body defines at least one lumen therethrough; and
a tissue anchoring assembly positioned near or at a distal end of the elongate body, wherein the tissue anchoring assembly is adapted to engage tissue within the hollow body organ upon the elongate body transitioning to the rigidized state and to release tissue upon the elongate body transitioning to the flexible state.

2. The system of claim 1 further comprising a handle assembly coupled to a proximal end of the elongate body adapted to actuate the elongate body between the flexible state and the rigidized state.

3. The system of claim 1 further comprising a second elongate body adapted to transition between a flexible state and a rigidized state and having a second tissue anchor assembly positioned near or art a distal end of the second elongate body, wherein the second elongate body is adapted to be slidably disposed within the at least one lumen defined through the elongate body.

4. The system of claim 1 further comprising an endoscope having a steerable distal portion adapted to be slidably disposed within the at least one lumen defined through the elongate body.

5. The system of claim 1 wherein the tissue anchoring assembly comprises a mesh member having an expanded anchoring configuration when engaging tissue and a low-profile delivery configuration when releasing tissue.

6. The system of claim 1 wherein the tissue anchoring assembly comprises at least one pivoting member having a pivoted anchoring configuration when engaging tissue and a low-profile delivery configuration when releasing tissue.

7. The system of claim 1 wherein the tissue anchoring assembly comprises an expandable malecot having a plurality of bendable members, wherein the malecot has a compressed configuration when engaging tissue and a low-profile delivery configuration when releasing tissue.

8. The system of claim 1 wherein the tissue anchoring assembly comprises a plurality of support members covered or coated with a membrane, wherein the support members have a radially expanded configuration when engaging tissue and a low-profile delivery configuration when releasing tissue.

9. The system of claim 1 wherein the tissue anchoring assembly comprises at least one expandable balloon having an expanded configuration when engaging tissue and a low-profile delivery configuration when releasing tissue.

10. The system of claim 9 wherein the at least one expandable balloon further comprises a membrane or coating adapted to increase frictional resistance against the tissue.

11. The system of claim 9 wherein the at least one expandable balloon is adapted to expand in off-axis relative to a longitudinal axis of the elongate body.

12. The system of claim 9 wherein the at least one expandable balloon is adapted to expand into a helical configuration.

13. The system of claim 1 wherein the tissue anchoring assembly comprises a plurality of expandable projections adapted to allow for uni-directional advancement of the elongate body through the hollow body organ.

14. The system of claim 1 wherein the elongate body comprises a steerable distal portion adapted to engage tissue when curved or angled relative to the hollow body organ and to release tissue when straightened relative to the hollow body organ.

15. A method for advancing an elongate instrument through a hollow body organ, comprising:
advancing an elongate body over an endoscope within the hollow body organ;
engaging a tissue region of the hollow body organ with a distal end of the endoscope;
pleating the engaged tissue onto an outer surface of the elongate body by proximally drawing the endoscope relative to the elongate body;
inhibiting the pleated tissue from unraveling from the elongate body; and
rigidizing the elongate body from a flexible state to a rigidized state while inhibiting the pleated tissue from unraveling.

16. The method of claim 15 wherein advancing an elongate body comprises advancing the elongate body within a colon or small intestines of a patient body.

17. The method of claim 15 wherein engaging a tissue region comprises configuring the distal end of the endoscope into a hook configuration.

18. The method of claim 15 wherein inhibiting the pleated tissue from unraveling comprises expanding a tissue anchoring assembly positioned near or at a distal end of the elongate body from a low-profile configuration to an expanded anchoring configuration.

19. The method of claim 18 wherein expanding a tissue anchoring assembly comprises expanding a mesh member.

20. The method of claim 18 wherein expanding a tissue anchoring assembly comprises expanding at least one balloon member.

21. The method of claim 20 wherein expanding at least one balloon member comprises expanding the balloon member in an off-axis configuration relative to the elongate body.

22. The method of claim 20 wherein expanding at least one balloon member comprises expanding the balloon member in a helical configuration.

23. The method of claim 20 wherein expanding at least one balloon member comprises expanding the balloon member having a covering or coating thereupon.

24. The method of claim 18 wherein expanding a tissue anchoring assembly comprises pivoting at least one member from a proximal-to-distal or distal-to-proximal direction.

25. The method of claim 15 wherein inhibiting the pleated tissue from unraveling comprises allowing the pleated tissue to be passed uni-directionally about the elongate body while preventing distal movement of the pleated tissue via a plurality of angled projections extending from the elongate body.

26. The method of claim 15 wherein inhibiting the pleated tissue from unraveling comprises steering a distal portion of the elongate body into contact against the pleated tissue.

27. The method of claim 15 further comprising transitioning the elongate body from the rigidized state to the flexible state and further advancing the elongate body within the hollow body organ.

28. A method for advancing an elongate instrument through a hollow body organ, comprising:
advancing an elongate body over an endoscope within the hollow body organ;
engaging a tissue region of the hollow body organ with a distal end of the endoscope;
pleating the engaged tissue onto an outer surface of the elongate body by proximally drawing the endoscope relative to the elongate body; and
steering a distal portion of the elongate body into contact against the pleated tissue to inhibit the pleated tissue from unraveling from the elongate body.

29. The method of claim 28 further comprising rigidizing the elongate body from a flexible state to a rigidized state while steering the distal portion of the elongate body into contact.

30. The method of claim 28 further comprising advancing the endoscope further within the hollow body organ relative to the elongate body.
